(19) United States
(12) Patent Application Publication
(10) Pub. No.: US 2017/0114531 A1
(43) Pub. Date: Apr. 27, 2017

(54) TOILET WITH AUTOMATIC URINE COLLECTING AND TESTING FUNCTION

(71) Applicant: Zhiqian YE, Baoshan Shanghai (CN)

(72) Inventor: Zhiqian YE, Baoshan Shanghai (CN)

(21) Appl. No.: 15/318,805

(22) PCT Filed: Jun. 9, 2015
(86) PCT No.: PCT/CN2015/081036

§ 371 Date: Dec. 14, 2016

(30) Foreign Application Priority Data
Jun. 16, 2014 (CN) 201420319010.7

(51) Int. Cl. E03D 11/02; G01N 33/493; A61B 10/00

(52) U.S. Cl. CPC E03D 11/02; A61B 10/007; G01N 33/493

(57) ABSTRACT

A toilet with automatic urine collecting and testing functions, comprising a toilet body and a urine testing device, the toilet body comprises a toilet bowl; the inner wall of the toilet bowl is provided with a section with a urine collecting groove thereon; the length of the urine collecting groove is one half to one fifth of the perimeter of the inner wall of the toilet bowl; one lower end of the urine collecting groove is provided with a urine collecting opening; the urine collecting opening is provided with a valve device therein; and the urine testing device comprises a urine dispensing device, a testing liquid dispensing device, a reaction device and a cleaning device. The product does not cause accumulation of urine, is convenient to clean, and realizes automatic urine collection and testing.

### BACKGROUND OF THE INVENTION

1. Field of the Invention

The present invention relates to medical instruments field, and more particularly to a toilet with automatic urine collecting and testing function.

2. Description of the Prior Art

The prior conventional urine biochemical test is generally conducted by professionals in hospitals and urine collecting should be operated by human so urine testing is very inconvenient.

The prior Chinese patent CN101787730B applied by the present inventor publicizes a toilet with intelligent automatic urine testing function. It can collect and test urine automatically but the structure of collecting urine is more complicated and it doesn't fully comply with the daily habit of people using toilets. Another prior Chinese patent CN102041844B applied by the present inventor publicizes an intelligently testing the structure of toilet urine collecting function. Though it complies with the daily habit of people using toilets, the structure of urine collecting is annular so it's easy to accumulate urine and inconvenient to clean. Besides, there is no urine testing device to match the urine collecting structure.

### BRIEF SUMMARY OF THE INVENTION

In order to overcome the technical problems, the present invention provides a toilet with automatic urine collecting and testing function.

In order to solve above problems, the present invention provides a toilet with automatic urine collecting and testing function, comprising a toilet body and a urine testing device, characterized in that the toilet body comprises a toilet bowl, the inner wall of the toilet bowl is provided with a section with a urine collecting groove thereon, the length of the urine collecting groove is one half to one fifth of the perimeter of the inner wall of the toilet bowl, one lower end of the urine collecting groove is provided with a urine collecting opening, the opening is provided with a valve device therein, a downward extending urine collecting tube is disposed under the urine collecting opening, the urine collecting tube is connected with urine collecting cup of urine testing device.

As a preferred embodiment, the present urine testing device comprises a urine dispensing device, the urine dispensing device comprising a urine dispensing metering pump, a urine dispensing slick, a drive screw and a drive motor, the drive motor driving the screw so the screw drives the urine dispensing slick to slide;

a testing liquid dispensing device, the testing liquid dispensing device comprising a plurality of testing liquid loading devices, a plurality of testing liquid delivery metering pump and a testing liquid dispensing pipe, the testing liquid dispensing pipe being provided with a plurality of testing liquid dispensing holes, one testing liquid loading device connecting one testing liquid delivery metering pump and one testing liquid dispensing hole;

a reaction device, the reaction device comprising a testing cup and a testing cup bracket, the testing cup bracket and the urine collecting cup being installed on the same rotation axle;

a cleaning device, the cleaning device comprising a cleaning pump, a sewage pump, a cleaning rotating motor and a cleaning pipe, the cleaning pipe is provided with a plurality of cleaning nozzle holes;

the urine dispensing slick and the testing liquid dispensing pipe being just provided above the testing cup bracket, the cleaning pipe being just provided under the testing cup bracket, the cleaning rotating motor driving the rotation axle to rotate the testing cup bracket and the urine collecting cup, when the testing is completed, the testing cup bracket and the urine collecting cup will be cleaned by the beneath cleaning nozzle hole.

The present invention has the advantages that 1) the urine collecting groove of the device is installed reasonably and is convenient to collect urine, does not cause accumulation of urine and is easy to clean; 2) the device and its assorted urine testing device are connected so it realizes automatic urine collecting and testing.

### BRIEF DESCRIPTION OF THE DRAWINGS

FIG. 1 is the top view of the toilet with automatic urine collecting and testing function.

FIG. 2 is the top view of another embodiment

FIG. 3 is the enlarged top view of the urine testing device.

FIG. 4 is the enlarged front view of the urine testing device.

REFERENCE NUMERALS
1—toilet body;
11—toilet bowl;
12a, 12b—urine collecting groove;
13—urine collecting open;
2—urine testing device;
21—urine collecting cup;
22—urine dispensing device;
221—urine dispensing metering pump;
222—urine dispensing slick;
223—drive screw;
224—drive motor;
23—testing liquid dispensing device;
231—testing liquid loading device;
232—testing liquid delivery metering pump;
233—testing liquid dispensing pipe;
234—testing liquid dispensing hole;
24—reaction device;
241—testing cup;
242—testing cup bracket;
25—cleaning device;
251—cleaning pump;
252—sewage pump;
253—cleaning rotating motor;
254—rotation axle;
255—cleaning pipe;
256—cleaning nozzle hole

### DETAILED DESCRIPTION OF THE PREFERRED EMBODIMENTS

The following text will further describe the present invention with reference to the accompanying drawings. The embodiment is only to explain the present invention but not to limit the technical solution.

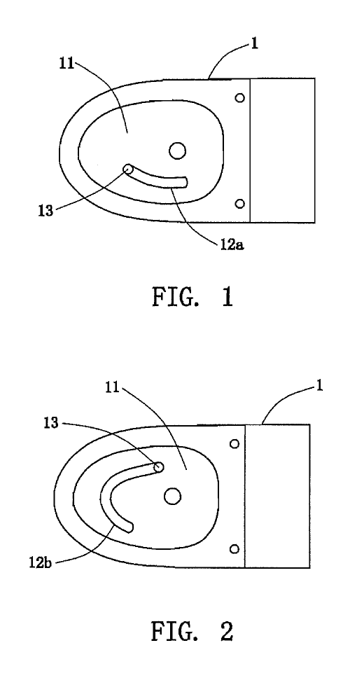

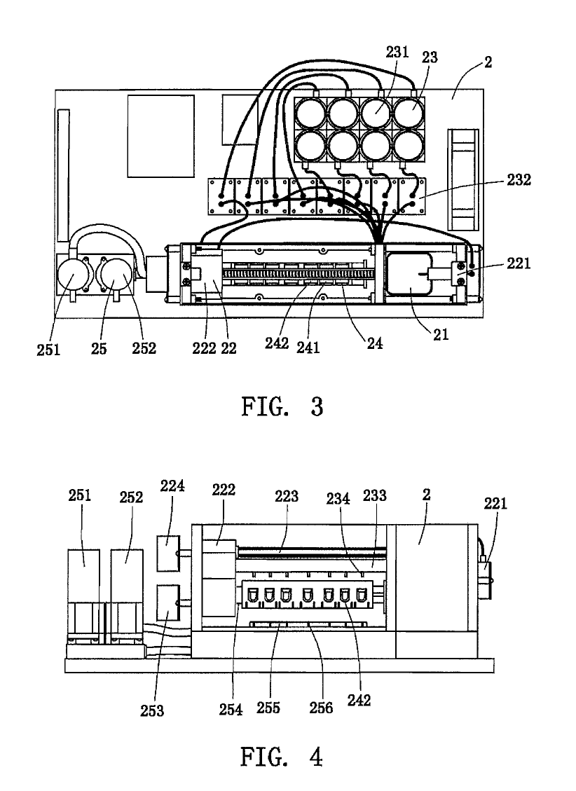

Please refer to FIG. 1 and FIG. 3, a toilet with automatic urine collecting and testing function comprises a toilet body 1 and a urine testing device 2; the toilet body 1 comprises a toilet bowl 11; the inner wall of the toilet bowl 11 is provided with a section with a urine collecting groove 12a thereon, the length of the urine collecting groove 12a is one half to one fifth of the perimeter of the inner wall of the toilet bowl. FIG. 1 is an embodiment of urine collecting groove 12a, the length of the urine collecting groove is one fifth of the perimeter of the inner wall of the toilet bowl. FIG. 2 is another embodiment of urine collecting groove, the length of the urine collecting groove is one half of the perimeter of the inner wall of the toilet bowl. The urine collecting groove can be provided on the position shown in FIG. 2 and can be provided on the other half of the semi-circle of the inner wall of the toilet bowl. One lower end of the urine collecting groove is provided with the urine collecting opening. When urine passes through the urine collecting groove, it will accumulate into the urine collecting groove and outflow through the lower urine collecting opening. Chinese patent CN102041844B publicizes an intelligently testing the structure of toilet urine collecting function. Though it complies with the daily habit of people using toilets, the structure of urine collecting is annular, surrounding around the toilet bowl with installment in 360 degree so it's easy to accumulate urine and inconvenient to clean. In fact, after long-term usage, there is no need to set the length of the urine collecting groove as a circle. Only randomly choosing one section can realize the urine collecting function and is not easy to cause accumulation of urine and easy to clean. One lower end of the urine collecting groove is provided urine collecting opening 13, the urine collecting opening 13 is provided with a value device therein (not shown in the figure), a downward extending urine collecting tube (not shown in the figure) is disposed under the urine collecting opening 13, the urine collecting tube is connected with urine collecting cup 21 of urine testing device 2.

Please refer to FIG. 3 and FIG. 4, urine testing device 2 comprises a urine collecting cup 21 and

a urine dispensing device 22, the urine dispensing device 22 comprising a urine dispensing metering pump 221, a urine dispensing slick 222, a drive screw 223 and a drive motor 224. The drive motor 224 drives the screw 223 so the screw 223 drives the urine dispensing slick 222 to slide;

a testing liquid dispensing device 23, the testing liquid dispensing device 23 comprising a plurality of testing liquid loading devices 231, a plurality of testing liquid delivery metering pump 232 and a testing liquid dispensing pipe 233, the testing liquid dispensing pipe 233 being provided with a plurality of testing liquid dispensing holes 234, one testing liquid loading device 231 connecting one testing liquid delivery metering pump 232 and one testing liquid dispensing hole 233;

a reaction device 24, the reaction device 24 comprising a testing cup 241 and a testing cup bracket 242. The testing cup bracket 242 and the urine collecting cup 21 are installed on the same rotation axle 254;

a cleaning device 25, the cleaning device 25 comprising a cleaning pump 251, a sewage pump 252, a cleaning rotating motor 253 and a cleaning pipe 255. The cleaning pipe 255 is provided with a plurality of cleaning nozzle holes 256.

The urine dispensing metering pump 221 sucks quantitative urine from the urine collecting cup 21 and drops it in turn into the testing cup 241 through gliding of the urine dispensing slick 222. The urine dispensing slick 222 and the testing liquid dispensing pipe 233 are just provided above the testing cup bracket 242. The cleaning pipe 255 is just provided under the testing cup bracket 242. The cleaning rotating motor 253 drives the rotation axle 254 to rotate the testing cup bracket 242 and the urine collecting cup 21. When the testing is completed, the testing cup bracket 242 and the urine collecting cup 21 will be cleaned by the beneath cleaning nozzle hole 256.

FIG. 4 is the front view of the rotating testing cup bracket 242 of the urine collecting and testing device 2. The testing cup bracket 242 and the urine collecting cup 21 can rotate in 360 degree. When cleaning, rotate the rotation axle 254 and keep the testing cup bracket 242 and the urine collecting cup 21 downwards facing the cleaning nozzle hole 256. Sewage will be drained through sewage pump 252.

The urine testing device has compact structure, reasonable layout and small size. When using in a toilet with urine collecting function, it can realize complete automation including automatic urine collecting, dispensing, testing and cleaning.

The above description is just the preferred embodiment of the present invention. Any person who skilled in the art may deform and modify the embodiment of the present invention within the spirit and scope of the appended claims, but these deformation and modification belong to the protection scope of the present invention.

What is claimed is:

1. A toilet with automatic urine collecting and testing function comprises a toilet body and a urine testing device, characterized in that the toilet body comprises a toilet bowl; the inner wall of the toilet bowl is provided with a section with a urine collecting groove thereon; the length of the urine collecting groove is one half to one fifth of the perimeter of the inner wall of the toilet bowl; one lower end of the urine collecting groove is provided with a urine collecting opening; the opening is provided with a valve device therein, a downward extending urine collecting tube is disposed under the urine collecting opening, the urine collecting tube is connected with urine collecting cup of urine testing device.

2. The toilet with automatic urine collecting and testing function as claimed in claim 1, characterizes in that, the urine testing device comprises a urine dispensing device, the urine dispensing device comprising a urine dispensing metering pump, a urine dispensing slick, a drive screw and a drive motor, the drive motor drives the screw so the screw drives the urine dispensing slick to slide;
a testing liquid dispensing device, the testing liquid dispensing device comprising a plurality of testing liquid loading devices, a plurality of testing liquid delivery metering pump and a testing liquid dispensing pipe, the testing liquid dispensing pipe being provided with a plurality of testing liquid dispensing holes, one testing liquid loading device connecting one testing liquid delivery metering pump and one testing liquid dispensing hole;
a reaction device, the reaction device comprising a testing cup and a testing cup bracket, the testing cup bracket and the urine collecting cup being installed on the same rotation axle;
a cleaning device, the cleaning device comprising a cleaning pump, a sewage pump, a cleaning rotating motor and a cleaning pipe, the cleaning pipe being provided with a plurality of cleaning nozzle holes;
the urine dispensing slick and the testing liquid dispensing pipe being provided above the testing cup bracket, the cleaning pipe being provided under the testing cup bracket, the cleaning rotating motor driving the rotation axle to rotate the testing cup bracket and the urine collecting cup, when the testing is completed, the testing cup bracket and the urine collecting cup will be cleaned by the beneath cleaning nozzle hole.
